(19) United States
(12) Patent Application Publication
(10) Pub. No.: US 2019/0374817 A1
(43) Pub. Date: Dec. 12, 2019

(54) SYSTEMS AND METHODS FOR PHYSICAL THERAPY USING AUGMENTED REALITY AND TREATMENT DATA COLLECTION AND ANALYSIS

(71) Applicant: SELFIT MEDICAL LTD, Ramat Hasharon (IL)

(72) Inventors: Almog NEUBERGER, Tel Aviv (IL); Avi Mordechai SHARIR, Ramat Hasharon (IL); Shahar FIGELMAN, Tel Aviv (IL); Amit KATZ, Gedera (IL)

(21) Appl. No.: 16/481,868

(22) PCT Filed: Feb. 7, 2018
(86) PCT No.: PCT/IL2018/050136

§ 371 Date: Jul. 30, 2019

(30) Foreign Application Priority Data
Mar. 22, 2017 (IL) 251340

(51) Int. Cl. A63B 24/00; A61B 5/11; A61B 5/0488; A61B 5/00; A63B 71/06

(52) U.S. Cl. CPC A63B 24/0075; A61B 5/1114; A61B 5/1128; A61B 5/0488; A61B 5/4023; A61B 5/6831; A61B 5/742; A61B 2505/09; A61B 2562/0247; A63B 2071/0655; A63B 2207/02; A63B 2024/0068; A61B 5/7455; A63B 71/0622

(57) ABSTRACT

The present invention relates to systems and methods for physical therapy in augmented reality (A R) comprising: at least one projector; at least one motion capture sensor; at least one surface E MG sensor, balance sensing unit; a computer in connection to said at least one projector, at least one motion capture sensor, and at least one surface E MG sensor and balance sensing unit. Wherein, movement instructions are provided to a patient in visual cues projected on a surface in patient's vicinity; and further wherein, data regarding patient's movements are collected by said at least one motion capture sensor and at least one surface E M G sensor and balance sensing unit. It is within the provision of the invention that said data is processed and analyzed into a representation of patient's status and progress, further providing suitable therapy and exercise plans.

### FIELD OF THE INVENTION

The present invention relates generally to the field systems and methods for physical therapy and rehabilitation. More specifically, the present invention relates to the field of physical therapy and rehabilitation systems and methods involving augmented reality and treatment feedback data collection and analysis.

### BACKGROUND OF THE INVENTION

Physical therapy aims to remedy physical impairment in patients and promotes better mobility and function through examination, movement exercise, and application of force.

Professionals in the field deal daily with various shortcomings that prevent them from maintaining a structured treatment plan, evaluation of patient status and progress, which may ultimately result in patient rehabilitation failure.

Traditionally, physiotherapists rely on their experience and have limited or no means to empirically evaluate patient's status and progress. This affects the professionals' ability to make consistent and accurate therapy plans and may ultimately result in waste of time and effort of both the patient and the therapist.

Additionally, physical therapy is inherently uncomfortable experience as it forces the patient outside of his comfort zone. This problem results in motivation problems in the patient which may ultimately cause the patient skip therapy sessions and reduce adherence of the rehabilitation process.

Several attempts have been made to address the above problems. One such attempt involves a virtual reality (VR) system that is comprised of a computer, a screen for displaying interactive and engaging content and a markerless motion capture sensor comparable to Microsoft Kinect™. A patient follows movement instructions displayed on the screen, while the system records and analyzes patient's movements. This way, the system follows patient's status and progress and allows the therapist some degree of control over patient rehabilitation process.

Yet, several shortcomings and deficiencies in the field still remain, for example:
a. Existing attempts collect only visual data regarding patient's movement. This type of data does not allow identifying and tracing the origin of the impairment in cases where nerve damage is involved (e.g. strokes). Analysis of only visual data does not allow verifying if a patient indeed tries to activate the correct muscles or tries to avoid activating the muscle because of pain or inability.
b. Patient's real body balance can't be determined based on visual data.
c. Existing attempts engage with patients via cues displayed on a screen. This prevents patients from engaging with the real physical world (i.e., floor, table) rather with virtual cue received in a displayed screen. Interacting with the real physical world is a seamless experience which reflects real world environment therefore places a lower barrier for the patient to adhere with the rehabilitation process.

In light of the above description of the current state of the art, it is clear that there is a long-standing need for a solution that employs a different approach to resolve the issues and deficiencies in existing attempted solutions in the field.

### SUMMARY OF THE INVENTION

The present invention relates to systems and methods for physical therapy in augmented reality (AR) comprising:
a. at least one projector;
b. at least one motion capture sensor;
c. at least one surface EMG sensor;
d. at least one balance sensor for determining patient's balance;
e. a computer in connection to said at least one projector, at least one motion capture sensor, and at least one surface EMG sensor, and a balance sensing unit;

Wherein, movement instructions are provided to a patient in visual cues projected on a surface in patient's vicinity; and further wherein, data regarding patient's movements are collected by said at least one motion capture sensor, at least one surface EMG sensor, and a balance sensing unit.

It is within the provision of the invention that said data is processed and analyzed into a representation of patient's status and progress, and further cross referenced with a data-base of representations of other patients' status and progress, further providing suitable therapy and exercise plans.

The present invention provides many advantages over the prior art, among others, the following:
a. The present invention collects not only visual data regarding patient's movement but also data regarding the electrical activity of the patient's muscles and patient's body balance. Analysis of this combined data feed allows identifying and tracing the origin of the impairment in cases where nerve damage is involved (e.g. strokes) or where the deficiency of patient's body balance is caused by asymmetry in the patient's body. This approach also allows a more accurate assessment and treatment of the impairment in a cost-effective manner.
b. Furthermore, analysis of a combination of visual with electrical muscle activity data allow verifying if a patient indeed tries to activate the correct muscles or tries to avoid the muscle because of pain or inability.
   For example, if a patient walks without correctly shifting his weight between strides, a system of the present invention will be capable to identify this deficiency even if the patient succeeds in reaching his designated goals.
c. The present invention does not engage patients through a two-dimensional screen where experience is limited and not seamless, but rather through a projector that is capable of projecting visual content on all surfaces in patient's vicinity. This opens new and unique uses and possibilities.
   For example, projecting foot print images on a floor instructing patients to perform specific strides at a chosen pace.
   Another example involves projecting the patient's image in a specific position on a wall, encouraging the patient to adapt to the position while providing a visual indication on the same wall regarding the patient's position in relation to the provided instructions.
   Another example involves projecting a virtual object on a table, encouraging the patient to move a physical object (e.g. a cup) from one place to another.

### BRIEF DESCRIPTION OF THE DRAWINGS

Embodiments and features of the present invention are described herein in conjunction with the following drawings:

FIG. 1 is a diagram depicting a system of the present invention.

FIG. 2 is a flowchart of a method of the present invention.

FIG. 3 is a flowchart of a method of the present invention.

FIG. 4 is a flowchart of a method of the present invention.

FIG. 5 is a diagram depicting an embodiment of a system of the present invention.

FIG. 6 is a flowchart of a method of the present invention.

FIG. 7 is a flowchart of a method of the present invention.

FIG. 8 is a diagram depicting a therapeutic exercise executed by an embodiment of the present invention.

FIG. 9 is a diagram depicting a therapeutic exercise executed by an embodiment of the present invention.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

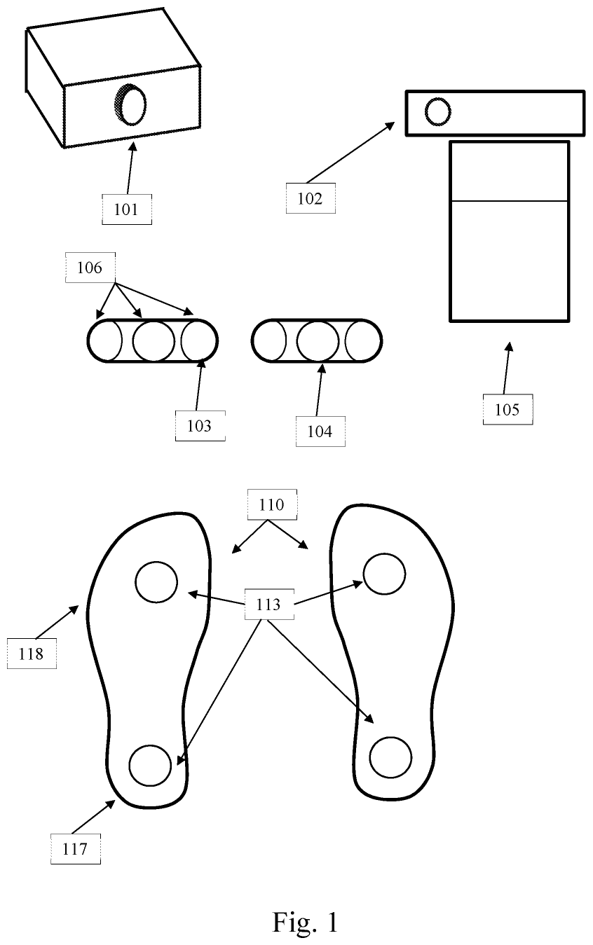

FIG. 1 depicts a system for physical therapy in augmented reality (AR) comprising:
a. one projector 101 that is capable of projecting visual cues on top of adjacent surfaces at normal interior lighting conditions; a BenQ MX620ST projector is suitable for this purpose but it should be mentioned that projectors of other types and makes may be also suitable to fill this purpose;
b. one motion capture sensor 102; a Microsoft Kinect™ sensor having 3d image capture capabilities is suitable for this purpose but it should be mentioned that other types and makes may be suitable to fill this purpose if they can detect movement and provide data regarding the patient's movements.
c. two surface EMG sensors 103, 104; This embodiment uses a limb band with a MyoWare™ EMG sensor coupled with several surface electrode pads 106 dispersed in the perimeter of said limb band.
   The surface EMG sensor may optionally further comprise a vibrating component (not shown) for providing feedback to a patient. Considering a patient wearing an EMG sensor with a vibrating component on each limb. If the system would determine that the patient is not properly activating a muscle in a certain limb, the system shall activate the vibrating component that is attached to the limb with the deficiency.
   The surface EMG sensor may optionally further LED lights (not shown) for providing feedback to a patient. Considering a patient wearing an EMG sensor with LED lights on each limb. If the system would determine that the patient is not properly activating a muscle in a certain limb, the system shall activate the LED lights that are attached to the limb with the deficiency.
d. a balance sensing unit 110;
   In this embodiment, the balance sensing unit is comprised of four pressure sensors 113 distributed evenly in 2 insoles for insertion in a patient's shoes.
   FlexiForce™ pressure sensors are suitable for this purpose but it should be mentioned that pressure sensors of other types and makes may be also suitable to fill this purpose.
   It should be mentioned that Tekscan iShoe™ insoles may be adapted for use in the system of the present invention without undue experimentation on-behalf of a person of average skill in the field of the present invention.
   One such pressure sensor is built into the heel of the insole 117 while the other pressure sensor is built into the fore of the insole 118. Each insole further comprises a battery, a microprocessor, a memory unit, and wireless communication module for transmitting collected data to the system (not shown).
   This configuration allows the inventive system to gather data regarding patient's weight distribution in movement.
e. a computer 105 in connection to said one projector, one motion capture sensor, two surface EMG sensors, and balance sensing unit;
   said computer may be a PC, a laptop, tablet or any computerized device capable of receiving input data from the sensors, making the necessary calculations for analyzing the raw data into a representation of patient's status and progress, comparing said representation of patient's status and progress to representations of other patients, further providing suitable therapy and exercise plans, and providing feedback through the projector and through other peripherals such as the vibrating component and the LED lights.

Movement instructions are provided to a patient in visual cues projected on a surface in patient's vicinity; while data regarding a patient's movements are collected by said motion capture sensor 102, the surface EMG sensors 103, 104, and balance sensing unit.

Among others, the system collects the following types of data: patient's movement in area, distance of patient relative to a certain point in area, EMG signals, balance, patient's stride length, number of steps per exercise, length of practice and etc.

The computer 105 may be connected by wire or by wireless network protocols such as Bluetooth and WIFI. The computer 105 that processes the data may be physically located at the same room as the sensors or may be a distant server that serves data from multiple users geographically distant from each other, at which case, a terminal computer is used for collecting the raw data, transmitting it for processing to the server computer, and for receiving and displaying the exercise/practice and performance data to user.

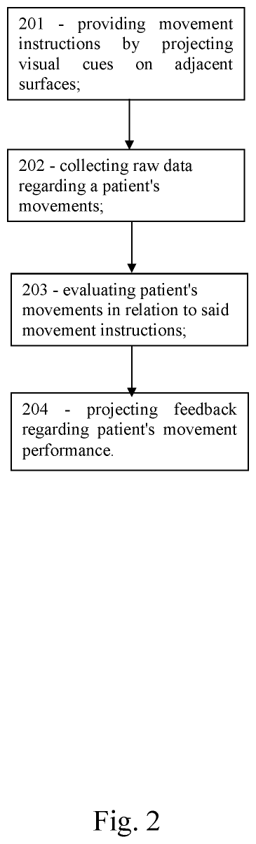

FIG. 2 depicts a method of an exemplary application for executing upon said computer, comprising steps of:
a. providing movement instructions by projecting visual cues on adjacent surfaces to a patient using said at least one projector 201;
b. collecting raw data regarding a patient's movements from said at least one motion capture sensor and at least one surface EMG sensor 202 balance sensing unit;
c. evaluating patient's movements in relation to said movement instructions 203;
d. projecting feedback regarding patient's performance 204 on adjacent surfaces.

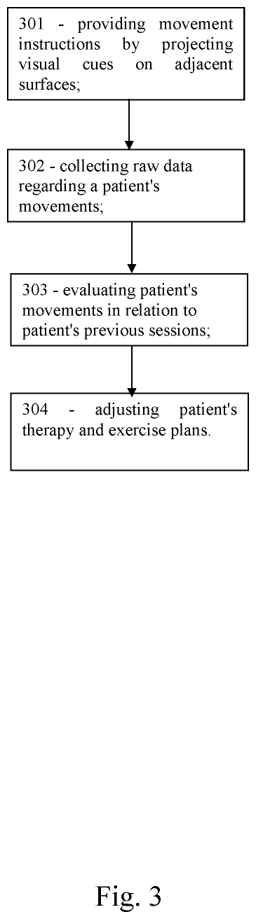

FIG. 3 depicts a method of an exemplary application for executing upon said computer, comprising steps of:
a. providing movement instructions by projecting visual cues on adjacent surfaces to a patient using said at least one projector 301;
b. collecting raw data regarding a patient's movements from said at least one motion capture sensor, at least one surface EMG sensor, and balance sensing unit 302;
c. evaluating patient's movements in relation to patient's previous sessions with the system 303;
d. adjusting patient's therapy and exercise plans 304.

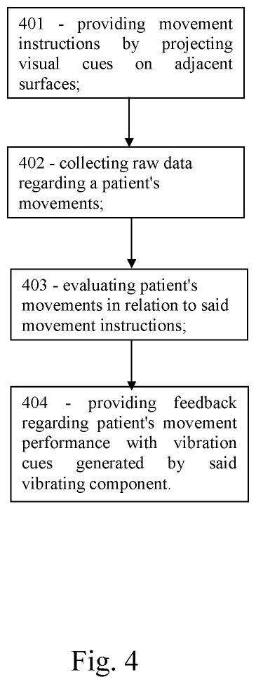

FIG. 4 depicts a method of an exemplary application for executing upon said computer, comprising steps of:
a. providing movement instructions by projecting visual cues on adjacent surfaces to a patient using said at least one projector 401;
b. collecting raw data regarding a patient's movements from said at least one motion capture sensor and at least one surface EMG sensor, balance sensing unit 402;
c. evaluating patient's movements in relation to said movement instructions 403;
d. providing feedback regarding patient's movement performance with vibration cues generated by said vibrating component 404.

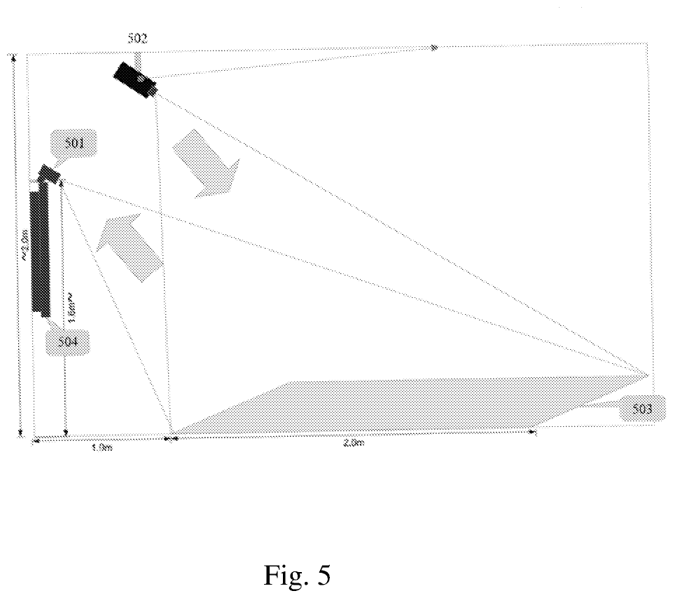

FIG. 5 depicts an exemplary configuration of a system consistent with the present invention. As can be seen the components of the system are distributed within a room. Presented is an exemplary configuration of a kinetic camera 501, and a projector 502.

In this preferred configuration both the projector and the kinetic camera are directed to a general direction of the interaction area 503 wherein, a patient is instructed by visual cues from the projector, and wherein the kinetic camera captures the movements of the patient.

In this exemplary configuration, the projector is configured to project on an interaction area of 200 cm length and 267 cm width. Using a BenQ MX620ST projector results in a 240 cm preferred throw range. The projector is also configured to an image brightness of 179 nits to provide sufficient luminosity in regular living room conditions.

Therefore, in this configuration, the projector is positioned directly on top of the closest edge of the interaction area in an approximate height of 2 meters. The kinetic camera on the other hand, is positioned horizontally 1 meter from the interaction area and 1.6 meters vertically therefrom.

This embodiment further comprises a screen 504 connected to the computer for presenting instructions and statistical data regarding the patient.

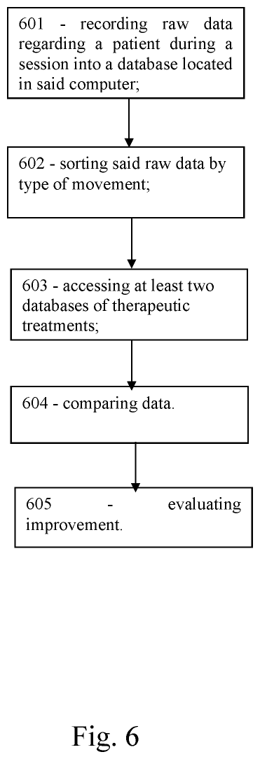

FIG. 6 depicts a method for evaluating patient's movements. The method is comprised the steps of:
a. recording raw data regarding a patient's movements during a therapeutic session into a database located in said computer 601;
b. sorting said raw data by type of movement 602;
   For example: raw data that was collected during an attempt to perform a step with left leg will be recorded in a single column of said database.
c. accessing at least two databases with recordings of therapeutic treatments of said patient 603;
   For example: accessing the database of the present treatment and the 4 last treatments.
d. comparing data recorded in accessed databases in chosen type of movement 604;
e. evaluating improvement in said chosen type of movement in comparison to previous recording of a therapeutic session 605;

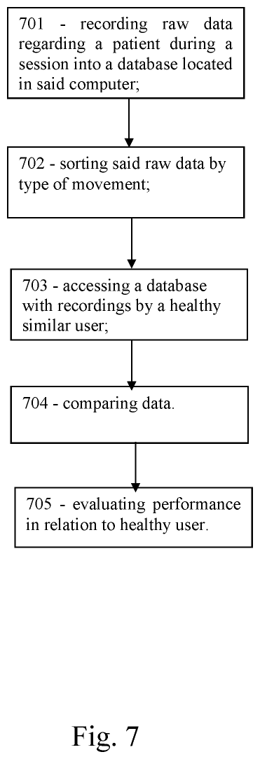

FIG. 7 depicts a method for evaluating patient's movements. The method is comprised the steps of:
a. recording raw data regarding a patient's movements during a therapeutic session into a database located in said computer 701;
b. sorting said raw data by type of movement 702;
c. accessing a database with recordings of type of movements by a healthy patient having similar characteristics (e.g. height, weight, age, gender) to said patient 703;
d. comparing data recorded in accessed databases in chosen type of movement 704;
e. evaluating performance of said patient in relation to healthy patient performing said chosen type of movement 705.

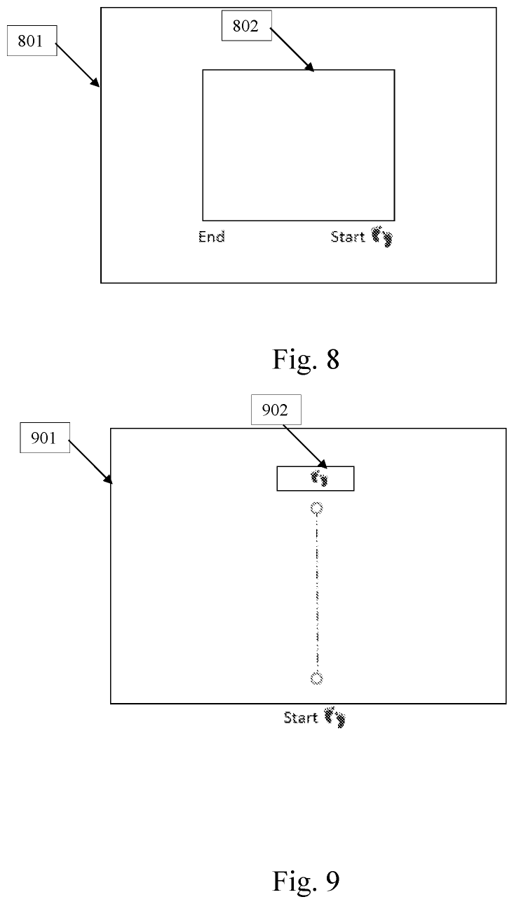

FIG. 8 depicts a projection of visual cues on an interaction area 801. The projector (not shown) projects an image of a square 802 on top of the interaction area 801. The patient is instructed through various audio-visual cues to walk along the lines of the square.

FIG. 9 depicts a projection of visual cues on an interaction area 901. Within the interaction area there is a stair 902. The projector (not shown) projects an image on top of the stair 802. The patient is instructed through various audio-visual cues to walk to the stair and step upon it.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

1. A system for physical therapy in augmented reality, comprising:
a. at least one projector;
b. at least one motion capture sensor;
c. at least one surface EMG sensor;
d. a balance sensing unit;
e. a computer in connection to said at least one projector, at least one motion capture sensor, and at least one surface EMG sensor, and a balance sensing unit;
wherein, movement instructions are provided to a patient in visual cues projected on a surface in patient's vicinity; and further wherein, data regarding patient's movements are collected by said at least one motion capture sensor and at least one surface EMG sensor, and balance sensing unit.

2. The system of claim 1, wherein said data is processed and analyzed into a representation of patient's status and progress, further providing suitable therapy and exercise plans.

3. The system of claim 1, wherein said surface EMG sensor is arranged in a limb band and comprises a plurality of surface electrode pads dispersed in the perimeter of said limb band.

4. The system of claim 3, wherein said surface EMG sensor further comprises a vibrating component for providing feedback to a patient.

5. The system of claim 3, wherein said surface EMG sensor further comprises LED lights for providing feedback to a patient.

6. A method for using a system of claim 1, comprising steps of:
a. providing movement instructions by projecting visual cues on adjacent surfaces to a patient using said at least one projector;
b. collecting raw data regarding a patient's movements from said at least one motion capture sensor, balance sensing unit, and at least one surface EMG sensor;
c. evaluating patient's movements in relation to said movement instructions;
d. providing feedback regarding patient's movement performance.

7. The method of claim 6 wherein the step of providing feedback is performed by projecting feedback on adjacent surfaces.

8. A method for using a system of claim 4, comprising steps of:
a. providing movement instructions by projecting visual cues on adjacent surfaces to a patient using said at least one projector;
b. collecting raw data regarding a patient's movements from said at least one motion capture sensor and at least one surface EMG sensor;
c. evaluating patient's movements in relation to said movement instructions;
d. providing feedback regarding patient's movement performance with vibration cues generated by said vibrating component.

9. A method for using a system of claim 5, comprising steps of:
a. providing movement instructions by projecting visual cues on adjacent surfaces to a patient using said at least one projector;
b. collecting raw data regarding a patient's movements from said at least one motion capture sensor and at least one surface EMG sensor;
c. evaluating patient's movements in relation to said movement instructions;
d. providing feedback regarding patient's movement performance with cues generated by said LED lights.

10. A method for using a system of claim 1, comprising steps of:
a. providing movement instructions by projecting visual cues on adjacent surfaces to a patient using said at least one projector;
b. collecting raw data regarding a patient's movements from said at least one motion capture sensor and at least one surface EMG sensor;
c. evaluating patient's movements;
d. adjusting patient's therapy and exercise plans.

11. The method of claim 10, wherein the step of evaluating patient's movement is comprised the steps of:
a. recording raw data regarding a patient's movements during a therapeutic session into a database located in said computer;
b. sorting said raw data by type of movement;
c. accessing at least two databases with recordings of previous therapeutic treatments of said patient;
d. comparing data recorded in accessed databases in chosen type of movement 604;
e. evaluating improvement in said chosen type of movement in comparison to previous recording of a therapeutic session.

12. The method of claim 10, wherein the step of evaluating patient's movement is comprised the steps of:
a. recording raw data regarding a patient's movements during a therapeutic session into a database located in said computer;
b. sorting said raw data by type of movement;
c. accessing a database with recordings of type of movements by a healthy patient having similar characteristics to said patient;
d. comparing data recorded in accessed databases in chosen type of movement;
e. evaluating performance of said patient in relation to healthy patient performing said chosen type of movement.
